SEQUENCE LISTING (4 sequences)

SEQ ID NO: 1 - DNA, 2151 bp, Homo sapiens
gcctctccaaaggctgcagaagtttcttgctaacaaaaagtccgcacattcgagcaaagacaggctttagcgagttattaaaaacttaggggcgctcttgtcccccacagggcccgaccgcacacagcaaggcgatggcccagctgtaagttggtagcactgagaactagcagcgcgcgcggagcccgctgagacttgaatcaatctggtctaacggtttcccctaaaccgctaggagccctcaatcggcgggacagcagggcgcgtcctctgccactctcgctccgaggtccccgcgccagagacgcagccgcgctcccaccacccacacccaccgcgccctcgttcgcctcttctccgggagccagtccgcgccaccgccgccgcccaggccatcgccaccctccgcagccatgtccaccaggtccgtgtcctcgtcctcctaccgcaggatgttcggcggcccgggcaccgcgagccggccgagctccagccggagctacgtgactacgtccacccgcacctacagcctgggcagcgcgctgcgccccagcaccagccgcagcctctacgcctcgtccccgggcggcgtgtatgccacgcgctcctctgccgtgcgcctgcggagcagcgtgcccggggtgcggctcctgcaggactcggtggacttctcgctggccgacgccatcaacaccgagttcaagaacacccgcaccaacgagaaggtggagctgcaggagctgaatgaccgcttcgccaactacatcgacaaggtgcgcttcctggagcagcagaataagatcctgctggccgagctcgagcagctcaagggccaaggcaagtcgcgcctgggggacctctacgaggaggagatgcgggagctgcgccggcaggtggaccagctaaccaacgacaaagcccgcgtcgaggtggagcgcgacaacctggccgaggacatcatgcgcctccgggagaaattgcaggaggagatgcttcagagagaggaagccgaaaacaccctgcaatctttcagacaggatgttgacaatgcgtctctggcacgtcttgaccttgaacgcaaagtggaatctttgcaagaagagattgcctttttgaagaaactccacgaagaggaaatccaggagctgcaggctcagattcaggaacagcatgtccaaatcgatgtggatgtttccaagcctgacctcacggctgccctgcgtgacgtacgtcagcaatatgaaagtgtggctgccaagaacctgcaggaggcagaagaatggtacaaatccaagtttgctgacctctctgaggctgccaaccggaacaatgacgccctgcgccaggcaaagcaggagtccactgagtaccggagacaggtgcagtccctcacctgtgaagtggatgcccttaaaggaaccaatgagtccctggaacgccagatgcgtgaaatggaagagaactttgccgttgaagctgctaactaccaagacactattggccgcctgcaggatgagattcagaatatgaaggaggaaatggctcgtcaccttcgtgaataccaagacctgctcaatgttaagatggcccttgacattgagattgccacctacaggaagctgctggaaggcgaggagagcaggatttctctgcctcttccaaacttttcctccctgaacctgagggaaactaatctggattcactccctctggttgatacccactcaaaaaggacacttctgattaagacggttgaaactagagatggacaggttatcaacgaaacttctcagcatcacgatgaccttgaataaaaattgcacacactcagtgcagcaatatattaccagcaagaataaaaaagaaatccatatcttaaagaaacagctttcaagtgcctttctgcagtttttcaggagcgcaagatagatttggaataggaataagctctagttcttaacaaccgacactcctacaagatttagaaaaaagtttacaacataatctagtttacagaaaaatcttgtgctagaatactttttaaaaggtattttgaataccattaaaactgcttttttttttccagcaagtatccaaccaacttggttctgcttcaataaatctttggaaaaactcaaaaaaaaaaaaaaa

SEQ ID NO: 2 - DNA, 1413 bp, Artificial Sequence
  Codon optimized sequence of vimentin
catatgagcacccgtagcgttagcgctagcgcttatcgtcgtatgtttggtggtccgggtacagcaagccgtccgagctctagccgtagctatgttaccacaagtacccgtacctatagcctgggtagcgcactgcgtccgagtaccagtcgtagcctgtatgcaagcagtccgggcggtgtttatgcaacccgtagctctgcagttcgtctgcgtagcagtgttccgggtgtgcgtctgttgcaggatagcgttgattttagcctggcagatgcaattaacaccgaattcaagaatacccgcaccaacgagaaagtggaactgcaagaactgaatgatcgctttgccaactatattgataaagtgcgttttctggaacagcaaaacaaaattctgttggcagaactggaacaactgaaaggtcagggtaaaagccgtctgggtgatctgtatgaagaggaaatgcgcgaactgcgtcgccaggttgatcagctgaccaatgataaagcacgtgttgaagttgaacgtgataatctggccgaagatattatgcgtctgcgtgagaaactgcaagaagagatgctgcagcgcgaagaggcagaaaacaccctgcagagctttcgtcaggatgttgataatgcaagcctggcacgtctggatctggaacgtaaagttgaaagcctgcaagaagagattgccttcctgaagaaactgcatgaagaggaaatccaagaactgcaggcacagattcaagaacagcatgttcagattgatgtggatgttagcaaaccggatctgaccgcggcactgcgtgatgttcgtcagcaatatgaaagcgttgcggcaaagaatctgcaagaggcagaagagtggtataaaagcaaattcgcagatctgagcgaagcagccaatcgtaacaatgatgccctgcgtcaggcaaaacaagaaagcaccgaatatcgtcgccaggttcagagcctgacctgtgaagttgatgcactgaaaggcaccaatgaatcactggaacgtcagatgcgtgaaatggaagagaattttgcagttgaagcagccaattatcaggataccattggtcgtctgcaggatgaaattcagaacatgaaagaagagatggcacgtcatctgcgtgaatatcaggatctgttgaatgttaaaatggccctggatattgaaattgccacctatcgtaaactgttggaaggtgaagagagccgtattagcctgccgctgccgaatttctcaagcctgaatctgcgcgaaaccaatctggatagtctgccgctggttgatacccatagcaaacgtaccctgttgattaaaaccgttgaaacccgtgatggtcaggtgattaatgaaaccagccagcatcacgatgacctggaataataagaatcc

SEQ ID NO: 3 - protein, 466 aa, Homo sapiens
MSTRSVSSSSYRRMFGGPGTASRPSSSRSYVTTSTRTYSLGSALRPSTSRSLYASSPGGVYATRSSAVRLRSSVPGVRLLQDSVDFSLADAINTEFKNTRTNEKVELQELNDRFANYIDKVRFLEQQNKILLAELEQLKGQGKSRLGDLYEEEMRELRRQVDQLTNDKARVEVERDNLAEDIMRLREKLQEEMLQREEAENTLQSFRQDVDNASLARLDLERKVESLQEEIAFLKKLHEEEIQELQAQIQEQHVQIDVDVSKPDLTAALRDVRQQYESVAAKNLQEAEEWYKSKFADLSEAANRNNDALRQAKQESTEYRRQVQSLTCEVDALKGTNESLERQMREMEENFAVEAANYQDTIGRLQDEIQNMKEEMARHLREYQDLLNVKMALDIEIATYRKLLEGEESRISLPLPNFSSLNLRETNLDSLPLVDTHSKRTLLIKTVETRDGQVINETSQHHDDLE

SEQ ID NO: 4 - protein, 144 aa, Homo sapiens
MKTLLLLAVIMIFGLLQAHGNLVNFHRMIKLTTGKEAALSYGFYGCHCGVGGRGSPKDATDRCCVTHDCCYKRLEKRGCGTKFLSYKFSNSGSRITCAKQDSCRSQLCECDKAAATCFARNKTTYNKKYQYYSNKHCRGSTPRC

(19) United States
(12) Patent Application Publication
(10) Pub. No.: US 2023/0270817 A1
(43) Pub. Date: Aug. 31, 2023

(54) VIMENTIN INHIBITORS

(71) Applicant: Filamon Pty Ltd, Melbourne (AU)

(72) Inventors: Bret CHURCH, Melbourne (AU); Ryung Rae KIM, Melbourne (AU); Timothy MANN, Melbourne (AU); Jun ZENG, Melbourne (AU); Kieran SCOTT, Melbourne (AU)

(21) Appl. No.: 18/016,983

(22) PCT Filed: Jul. 22, 2021
(86) PCT No.: PCT/AU2021/050791

§ 371 Date: Jan. 19, 2023

(30) Foreign Application Priority Data
Jul. 22, 2020 (AU) 2020902552

(51) Int. Cl. A61K 38/12; G01N 33/88

(52) U.S. Cl. CPC A61K 38/12; G01N 33/88

(57) ABSTRACT

The present disclosure relates to methods of inhibiting vimentin activity, methods of screening for new vimentin inhibitors and uses of new vimentin inhibitors.

### CROSS-REFERENCE TO RELATED APPLICATIONS

The present application claims priority from Australian Provisional Patent Application No. 2020902552 filed on 22 Jul. 2020, the contents of which are incorporated herein by reference in their entirety.

### TECHNICAL FIELD

The present disclosure relates to methods of inhibiting vimentin activity, methods of screening for new vimentin inhibitors and uses of new vimentin inhibitors.

### BACKGROUND

Vimentin is a structural protein expressed in mammalian cells and is the major cytoskeletal component of mesenchymal cells. As well as its role as a scaffold protein, vimentin is thought to play a role in a number of stress-induced signalling pathways.

Co-localisation of vimentin and hGIIA (also known as human sPLA₂-IIA, human group IIA secreted phospholipase A₂ and the protein encoded by the human PLA2G2A gene) has been observed during the occurrence of arachidonic acid metabolism in the context of synovial inflammation (Lee et al., J Biol Chem. May 24; 288(21): 15269-15279; 2013). Arachidonic acid has been suggested as playing a role in the synthesis of downstream inflammatory metabolites. However, the precise molecular interactions that define vimentin's role in inflammation and cell proliferation have not previously been elucidated.

### SUMMARY

The present disclosure is based in part on the surprising finding that vimentin binds directly to hGIIA and on the demonstration that compounds can effectively inhibit vimentin's functions by inhibiting the binding interaction between vimentin and hGIIA. On the basis of these findings, the inventors have developed new methods of screening for vimentin inhibitors, methods of inhibiting vimentin activity, and methods of treating or preventing a disease, disorder or condition involving a vimentin mediated pathway.

Accordingly, the present disclosure provides a method of inhibiting vimentin and/or hGIIA, the method comprising inhibiting the binding of vimentin to hGIIA at coil 2 of vimentin. The method may comprise administering an inhibitor of the binding of vimentin to hGIIA at coil 2 of vimentin.

In one particular form, the present disclosure provides a method of treating or preventing a disease, disorder or condition involving a vimentin-mediated pathway and/or a hGIIA-mediated pathway in a subject having or susceptible to the disease, disorder or condition.

The present disclosure also provides the use of an inhibitor of the binding of vimentin to hGIIA at coil 2 of vimentin in the manufacture of a medicament for the treatment of a disease, disorder or condition involving a vimentin-mediated pathway and/or a hGIIA-mediated pathway.

In addition, the present disclosure provides an inhibitor of the binding of vimentin to hGIIA at coil 2 of vimentin, for use in treating a disease, disorder or condition involving a vimentin-mediated pathway and/or a hGIIA-mediated pathway.

In another aspect, the present disclosure provides a number of screening methods for identifying vimentin inhibitors. For example, the present disclosure provides a method of screening for an inhibitor of the vimentin-hGIIA interaction, comprising identifying an agent that inhibits the direct physical binding of vimentin to hGIIA. The method may comprise identifying an agent that inhibits the binding of vimentin to hGIIA at coil 2 of vimentin. As disclosed herein, vimentin inhibitors that bind to coil 2 have been shown to be particularly effective in inhibiting the binding of vimentin to hGIIA.

The present disclosure also provides a method of identifying an agent that inhibits the vimentin-hGIIA interaction, comprising contacting coil 2 of vimentin with a test agent, wherein if the test agent binds to coil 2 of vimentin, the test agent is identified as an agent that inhibits the vimentin-hGIIA interaction.

In addition, the present disclosure provides a method of screening for a potential inhibitor of inflammation, cellular proliferation and/or epithelial-to-mesenchymal transition, the method comprising determining whether a test agent inhibits the binding of vimentin to hGIIA at coil 2 of vimentin, wherein if the test agent inhibits the binding of vimentin to hGIIA at coil 2 of vimentin, the test agent is identified as a potential inhibitor of inflammation, cellular proliferation and/or epithelial-to-mesenchymal transition. Vimentin has been shown to play a role in inducing each of inflammation, EMT and cellular proliferation, and the inhibition of vimentin as disclosed herein therefore provides a mechanism for inhibiting each of inflammation, EMT and cellular proliferation.

In another aspect, the present disclosure provides an inhibitor of the vimentin-hGIIA interaction, obtained by any of the screening methods disclosed herein. Furthermore, any of the methods of inhibiting the binding of vimentin to hGIIA at coil 2 of vimentin disclosed herein, can be performed using an inhibitor of the vimentin-hGIIA interaction that is obtained by any of the screening methods disclosed herein.

In yet another aspect, the present disclosure provides an inhibitor of the vimentin-hGIIA interaction, wherein the inhibitor binds to residues 347 to 357 and/or 342 to 350 in coil 2 of vimentin, and wherein the inhibitor is not the cyclic peptide cyclo-((2-Nal)-Leu-Ser-(2-Nal)-Arg).

Specific embodiments of each aspect of the invention are described, for example, in the dependent claims.

### BRIEF DESCRIPTION OF THE DRAWINGS

The following figures form part of the present specification and are included to further demonstrate certain aspects of the present disclosure. The disclosure may be better understood by reference to one or more of these figures in combination with the detailed description of specific embodiments presented herein.

FIG. 1. The anti-prostate cancer drug, c2, binds directly to vimentin, thereby blocking hGIIA-mediated tumourigenesis. A. c2 potently slows the growth of prostate cancer (LNCaP tumours) in a mouse xenograft model. B. hGIIA colocalises with vimentin in prostate cancer cell lines. Red, vimentin immunofluorescence; green hGIIA immunofluorescence; yellow colocalisation. Left image, PC-3 cells, right image LNCaP cells. C. hGIIA. binds directly to vimentin in LNCaP cells as demonstrated by (left image) Forster resonance energy transfer (FRET), pink, areas of direct binding resonances between vimentin and hGIIA, captured using Fluorescence lifetime imaging microscopy (FLIM). Vimentin (donor) and hGIIA (acceptor) were imaged using antibodies conjugated to Alexa fluor 488 and Alexa fluor 568, respectively. (right image) a phasor plot of fluorescent lifetimes used to calculate FRET efficiency.

FIG. 2. Coil 2 of Vimentin binds directly to hGIIA in vitro. A. Full length vimentin (blue), coil Wed) or coil 2 (green), bound to 96-well plates, was incubated with hGIIA at concentrations shown. hGIIA binding was detected with alkaline phosphatase-labelled hGIIA-specific monoclonal antibody), B. Top panel: Full length vimentin was incubated with hGIIA in the presence of c2 (blue) or the hGIIA inhibitor LY311727 (red) as shown and hGIIA binding detected as above. B Bottom panel: coil 2 of vimentin was incubated with hG11A in the presence of c2 (blue) or the hGIIA inhibitor LY311727 (red) as shown and hGIIA binding detected as above. C. 3H-labelled c2 binds weakly to hGILA and more strongly to full-length and coil 2 of vimentin, but not to coil 1 in the absence of hGI1A. D & E. in silky) docking experiments using a 10 ns molecular dynamics simulation of c2 to vimentin dimer (PDB code 1gk4) identify energetically favourable c2 binding conformations and vimentin binding sites. c2 structure is represented by coloured sticks and vimentin is represented by a space-filling model in (D) and a cartoon ribbon representation of the alpha-helices thereof (E). Key vimentin amino acids involved in the c2 interaction are identified as thin sticks and labels (E).

FIG. 3. Docking of c2 on vimentin by a second method. A. Docking was conducted on multiple grids (magenta) to include the entire protein surface area of vimentin coil 1 (A) and 2 (B). B. The top 10 scoring poses of c2 (sticks) docked to the coil 2 of vimentin (white cartoon) by the method described in FIG. 3A. The 13 residues that are unique to vimentin among the group III intermediate filament proteins are depicted in spheres.

FIG. 4. Summary of c2 binding site on vimentin coil 2. A. The amino acid residues representing sites of binding to the inhibitor c2 are illustrated, based on molecular modelling studies. Those contact residues identified with modelling strategy 1 (FIG. 2D, FIG. 2E) are bolded and underscored. Those contact residues identified by modelling strategy 2 are bolded and overscored.

### KEY TO THE SEQUENCE LISTING

SEQ ID NO: 1 Nucleotide sequence for a reference human vimentin gene (RefSeq NM_003380.3).
SEQ ID NO: 2 Nucleotide sequence for a codon optimized human vimentin gene.
SEQ ID NO: 3 Amino acid sequence for a reference human vimentin protein (Uniprot accession no. P08670).
SEQ ID NO: 4 Amino acid sequence for a reference human hGIIA protein (Uniprot accession no. P14555).

### DETAILED DESCRIPTION

### General Techniques and Definitions

Unless otherwise defined herein, technical and scientific terms used herein shall have the meanings that are commonly understood by those of ordinary skill in the art (e.g., in immunology, immunohistochemistry, protein chemistry, cell biology, biochemistry and chemistry).

With regard to the definitions provided herein, unless stated otherwise, or implicit from context, the defined terms and phrases include the provided meanings. Unless explicitly stated otherwise, or apparent from context, the terms and phrases below do not exclude the meaning that the term or phrase has acquired by a person skilled in the relevant art.

The definitions are provided to aid in describing particular embodiments, and are not intended to limit the claimed invention, because the scope of the invention is limited only by the claims.

The present disclosure is not to be limited in scope by the specific embodiments described herein, which are intended for the purpose of exemplification only. Functionally equivalent products, compositions and methods are clearly within the scope of the disclosure, as described herein.

Each feature of any particular aspect or embodiment of the present disclosure may be applied mutatis mutandis to any other aspect or embodiment of the present disclosure unless specifically stated otherwise.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Unless otherwise required by context, singular terms shall include pluralities and plural terms shall include the singular. Thus as used herein, the singular forms of “a”, “an” and “the” include plural forms of these words, unless the context clearly dictates otherwise. For example, a reference to “a bacterium” includes a plurality of such bacteria, a reference to “a cell” includes populations of a plurality of cells. and a reference to “an allergen” is a reference to one or more allergens.

The term “and/or”, e.g., “X and/or Y” shall be understood to mean either “X and Y” or “X or Y” and shall be taken to provide explicit support for both meanings or for either meaning.

The terms “administration of” and or “administering a” compound should be understood to mean providing a compound or inhibitor disclosed herein to a test organism (such as a mammalian cell or a non-human animal model), or to an individual (such as a mammal, or, in a more specific example, a human) in need of treatment.

Throughout this specification, the word “comprise” or variations such as “comprises” or “comprising” will be understood to imply the inclusion of a stated element, integer or step, or group of elements, integers or steps, but not the exclusion of any other element, integer or step, or group of elements, integers or steps.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations of any two or more of said steps or features.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

It will be clearly understood that, although a number of prior art publications are referred to herein, this reference does not constitute an admission that any of these documents forms part of the common general knowledge in the art, in Australia or in any other country.

### Methods of Treating

As used herein, the terms “treating”, “treat” or “treatment” and variations thereof, refer to clinical intervention designed to alter the natural course of the individual or cell being treated during the course of clinical pathology. Desirable effects of treatment include decreasing the rate of disease progression, ameliorating or palliating the disease state, and remission or improved prognosis.

As used herein, the terms “prevent”, “prevented”, or “preventing”, refer to a prophylactic treatment which increases the resistance of a subject to developing the disease or condition or, in other words, decreases the likelihood that the subject will develop the disease or condition as well as a treatment after the disease or condition has begun in order to reduce or eliminate it altogether or prevent it from becoming worse. These terms also include within their scope preventing the disease or condition from occurring in a subject which may be predisposed to the disease or condition but has not yet been diagnosed as having it.

As used herein, the term “subject” refers to any animal, for example, a mammalian animal, including, but not limited to humans, non-human primates, livestock (e.g., sheep, horses, cattle, pigs, donkeys), companion animals (e.g., pets such as dogs and cats), laboratory test animals (e.g., mice, rabbits, rats, guinea pigs), performance animals (e.g., racehorses, camels, greyhounds) or captive wild animals. In various examples, the “subject” is a human. Typically, the terms “subject” and “patient” are used interchangeably, particularly in reference to a human subject.

As used herein, the term “test organism” refers to any cell or organism that may be used to determine the function or binding of vimentin and/or hGIIA as disclosed herein. The cell may be derived from any animal, for example, a mammalian animal, including, but not limited to humans, non-human primates, livestock (e.g., sheep, horses, cattle, pigs, donkeys), companion animals (e.g., pets such as dogs and cats), laboratory test animals (e.g., mice, rabbits, rats, guinea pigs), performance animals (e.g., racehorses, camels, greyhounds) or captive wild animals. The test organism may be any such animal.

The methods disclosed herein may be a method of treating or preventing a disease, disorder or condition involving a vimentin-mediated pathway and/or an hGIIA-mediated pathway, in a subject having or susceptible to the disease, disorder or condition. Accordingly, in a particular form, the present disclosure provides a method of treating or preventing a disease, disorder or condition involving a vimentin-mediated pathway and an hGIIA-mediated pathway, in a subject having or susceptible to the disease, disorder or condition. In another form, the present disclosure provides a method of treating or preventing a disease, disorder or condition involving a vimentin-mediated pathway, in a subject having or susceptible to the disease, disorder or condition. In a further form, the present disclosure provides a method of treating or preventing a disease, disorder or condition involving an hGIIA-mediated pathway, in a subject having or susceptible to the disease, disorder or condition.

In some examples, the disease, disorder or condition may be a disease, disorder or condition at least partly caused or mediated by hGIIA-mediated inflammation. Examples of a disease, disorder or condition involving a vimentin mediated pathway include, but are not limited to: cataracts, cancer, Crohn's disease, rheumatoid arthritis, myopathies, inflammatory bowel disease, asthma, coronavirus infection, atherosclerosis, chronic pain and HIV infection in a subject. Other examples, as well as methods for determining whether a disease, disorder or condition are mediated by vimentin and/or hGIIA are known in the art.

Methods of inhibiting vimentin and/or hGIIA Vimentin is a type III intermediate filament (IF) protein that is expressed in mammalian cells including mesenchymal cells. IF proteins are found in all animal cells and bacterial cells. IFs, along with tubulin-based microtubules and actin-based microfilaments, comprise the cytoskeleton. Vimentin is the major cytoskeletal component of mesenchymal cells. Thus, vimentin is often used as a marker of mesenchymal-derived cells or cells undergoing an epithelial-to-mesenchymal transition (EMT) during both normal development and metastatic progression.

The vimentin protein comprises a number of different domains. For example, the structural organization of a vimentin monomer comprises a central, mostly α-helical “rod” domain flanked by intrinsically disordered non-α-helical N-terminal (“head”) and C-terminal (“tail”) domains. The rod consists of two equally sized α-helical subdomains termed coil 1 (146 amino acids) and coil 2 (140 amino acids), which are connected by the 16 amino acid-long non-α-helical linker segment L12. Coil 1 is divided into a short coil 1A and a longer coil 1B segment. Linker L1 connecting the coil 1A and B subdomains is 8 amino acids long and evolutionarily highly conserved; similar to other intrinsically disordered domains, it may optionally form a distinct structure. Indeed, in the crystal of a larger fragment derived from coil 1, linker L1 adopts an α-helical fold without being involved in the coiled-coil formation of coil 1A and coil 1B. Coil 2 represents a continuous α-helix in which the first 35 amino acids form hendecad repeats establishing a right-handed helix with a very large pitch. Hence, in the dimer, the two chains essentially form parallel helices, which are designated as “paired bundle” or pb (Premchandar et al., 2016 J Biol Chem 291(48):24931-24950).

Coil 2 of vimentin may be defined as comprising or consisting of the amino acids from positions 254 to 405 of SEQ ID NO: 3. Alternatively, coil 2 of vimentin may be defined as comprising or consisting of the 140 amino acids from positions 265-404 as defined in Premchandar et al., 2016. Alternatively, coil 2 of vimentin may be defined as comprising or consisting of amino acids from positions 265-405 as defined in Premchandar et al., 2016. As a further alternative, coil 2 of vimentin may be defined as a coil region of vimentin comprising amino acid residues 347 to 357 as set out in SEQ ID NO: 3. As a further alternative, coil 2 of vimentin may be defined as a coil region of vimentin comprising amino acid residues 342 to 350 as set out in SEQ ID NO: 3. As a further alternative, coil 2 of vimentin may be defined as a coil region of vimentin comprising any one or more of the following amino acids according to the amino acid numbering set out in SEQ ID NO: 3: M347, N350, F351, V353, E354 or N357. In certain examples, coil 2 of vimentin may be defined as a coil region of vimentin comprising any one or more of the following amino acids according to the amino acid numbering set out in SEQ ID NO: 3: M347, N350, V353 or N357. In other examples, coil 2 of vimentin may be defined as a coil region of vimentin comprising any one or more of the following amino acids according to the amino acid numbering set out in SEQ ID NO: 3: M347, F351 and E354.

Coil 1 of vimentin may be defined as comprising or consisting of the amino acids from positions 103-249 as defined in Premchandar et al., 2016.

Vimentin has been shown to play a role in supporting and anchoring the position of organelles in the cytosol. Vimentin may be attached to the nucleus, endoplasmic reticulum, and mitochondria, either laterally or terminally.

Amongst other functions, vimentin is responsible for maintaining cell shape, integrity of the cytoplasm, and stabilizing cytoskeletal interactions. Although transgenic mice that lack vimentin appear normal and did not show functional differences, (Colucci-Guyon E et al., 1994. “Mice lacking vimentin develop and reproduce without an obvious phenotype”. Cell. 79 (4): 679-94), wounded mice that lack the vimentin gene heal slower than their wild type counterparts (Eckes B et al., 2000. “Impaired wound healing in embryonic and adult mice lacking vimentin”. Journal of Cell Science. 113 (13): 2455-62.

Vimentin has been suggested as interacting with human group IIA-secreted phospholipase A2 (hGIIA) (although the precise nature of the interaction has not previously been elucidated) to mediate the release of arachidonic acid, an important molecule for the synthesis of inflammatory metabolites. Without wishing to be bound by theory, it is believed that the disruption of the vimentin-hGIIA interaction as disclosed herein reduces inflammation and cell proliferation.

As used herein, the term “inhibit” shall be taken to mean hinder, reduce, restrain or prevent vimentin activity in a vimentin protein relative to vimentin activity in a vimentin protein in a cell in which the vimentin-hGIIA interaction is intact. Thus, as used herein, the inhibition of vimentin may comprise inhibition of vimentin activity.

Vimentin activity and/or hGIIA activity may be inhibited in any measurable amount. Inhibition of vimentin activity and/or hGIIA activity may be complete or may be partial. Thus, the methods disclosed herein may comprise at least partial inhibition of vimentin activity and/or hGIIA activity. For example, the activity of vimentin and/or hGIIA may be reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or 100% following disruption of the vimentin-hGIIA interaction (e.g., relative to the same measurement of activity before disruption of the vimentin-hGIIA interaction).

Vimentin activity and/or hGIIA activity may be measured through any suitable means known in the art, or any of the methods disclosed herein. For example, vimentin activity and/or hGIIA activity may be measured through assays which measure the level of one or more inflammatory markers, such as inflammatory cytokines, inflammatory prostaglandins or the proliferation of prostate cancer cells. In another example, the level of binding of vimentin to hGIIA may be measured as a proxy for another activity of vimentin. Any suitable methods for determining the nature of the binding interaction between vimentin and hGIIA may be used.

Typically, the inhibitory activity of candidate or test agents may be assessed by in vitro and/or in vivo assays that detect or measure vimentin activity and/or hGIIA activity in the presence of the test agent. Accordingly, the screening methods described herein may include the initial step of contacting coil 2 of vimentin, such as expressed within a suitable test cell or animal, with an effective amount of a test or candidate agent. Suitably, if the test agent binds to coil 2 of vimentin, the test agent is identified as an agent that inhibits the interaction of vimentin with hGIIA.

At the clinical level, screening a candidate agent may include obtaining samples from test subjects before and after the subjects have been exposed to a test compound. The levels of vimentin activity and/or hGIIA activity in the samples may then be measured and analysed to determine whether the levels and/or activity thereof changes after exposure to a candidate agent. By way of example, protein product levels in the samples may be determined by mass spectrometry, western blot, ELISA, electrochemistry and/or by any other appropriate means known to one of skill in the art.

It will be understood by a person skilled in the art that inhibition may be achieved at the protein level. The inhibitor may be a direct inhibitor of the vimentin-hGIIA interaction. For example, the inhibitor may bind to vimentin to inhibit its function by changing its conformation or by affecting its binding site such that it is no longer able to bind to hGIIA. In another example, the inhibitor may bind to hGIIA to inhibit its function by changing its conformation or by affecting its binding site such that it is no longer able to bind to vimentin. Any inhibitor such as those disclosed herein may be capable of disrupting the vimentin-hGIIA interaction such that the endogenous function of the vimentin-hGIIA complex is inhibited.

The inhibitor disclosed herein may bind to coil 2 of vimentin. Thus, the inhibitor may bind to any one or more amino acids within coil 2 as defined herein. Accordingly, the inhibitor may bind to any one or more amino acids within positions 254 to 405 of SEQ ID NO: 3. Alternatively, the inhibitor may bind to any one or more amino acids within the 140 amino acids from positions 265-404 as defined in Premchandar et al., 2016. Alternatively, the inhibitor may bind to any one or more amino acids within the amino acids from positions 265-405 as defined in Premchandar et al., 2016. Alternatively, the inhibitor may bind to any one or more amino acids within a coil region of vimentin comprising amino acid residues 347 to 357 as set out in SEQ ID NO: 3. Alternatively, the inhibitor may bind to any one or more amino acids within a coil region of vimentin comprising amino acid residues 342 to 350 as set out in SEQ ID NO: 3. Alternatively, the inhibitor may bind to any one or more amino acids within a coil region of vimentin comprising any one or more of the following amino acids according to the amino acid numbering set out in SEQ ID NO: 3: M347, N350, F351, V353, E354 or N357. Alternatively, the inhibitor may bind to any one or more amino acids within a coil region of vimentin comprising any one or more of the following amino acids according to the amino acid numbering set out in SEQ ID NO: 3: M347, N350, V353 or N357. Alternatively, the inhibitor may bind to any one or more amino acids within a coil region of vimentin comprising any one or more of the following amino acids according to the amino acid numbering set out in SEQ ID NO: 3: M347, F351 and E354. Alternatively, the inhibitor may bind to at least M347, F351 and E354 of SEQ ID NO: 3.

The inhibitor may be any one or more of a nucleic acid inhibitor, a small molecule inhibitor, a protein inhibitor or a peptide inhibitor.

In one example, the inhibitor may be a nucleic acid inhibitor of vimentin or of hGIIA. For example, the inhibitor may be an aptamer (adaptable oligomer). Aptamers are single stranded oligonucleotides or oligonucleotide analogues that are capable of forming a secondary and/or tertiary structure that provides the ability to bind to a particular target molecule, such as a protein or a small molecule, e.g., vimentin or hGIIA. Thus, aptamers are considered the oligonucleotide analogy to antibodies. In general, aptamers comprise about 15 to about 100 nucleotides, such as about 15 to about 40 nucleotides, for example about 20 to about 40 nucleotides, since oligonucleotides of a length that falls within these ranges can be prepared by conventional techniques.

An aptamer can be isolated from or identified from a library of aptamers. An aptamer library is produced, for example, by cloning random oligonucleotides into a vector (or an expression vector in the case of an RNA aptamer), wherein the random sequence is flanked by known sequences that provide the site of binding for PCR primers. An aptamer that provides the desired biological activity (e.g., binds specifically to vimentin and/or hGIIA) is selected. An aptamer with increased activity is selected, for example, using SELEX (Systematic Evolution of Ligands by EXponential enrichment). Suitable methods for producing and/or screening an aptamer library are described, for example, in Elloington and Szostak, Nature 346:818-22, 1990; U.S. Pat. No. 5,270,163; and/or U.S. Pat. No. 5,475,096.

A reference nucleotide sequence of vimentin is provided in SEQ ID NO: 1 and reference amino acid sequences for vimentin and hGIIA are provided in SEQ ID NOs 3 and 4, respectively. The mature sequence of hGIIA is believed to begin at residue M11 of the sequence provided in SEQ ID NO: 4.

In another example, the inhibitor is a peptide inhibitor. The peptide inhibitor may be a cyclic peptide. For example, the peptide inhibitor may be a cyclic peptide of the following formula:
A1-A2-A3-A4-A5
in which:
A1 is F or Y or W or 2NapA;
A2 is L or I;
A3 is S or T;
A4 is F or Y or W or 2NapA; and
A5 is R or K.

In one example, the peptide inhibitor is selected from the group consisting of cFLSYK, cFLSYR and c(2NapA)LS(2NapA)R. When used herein the term “cFLSYK” means “cyclic FLSYK”, “cFLSYR” means “cyclic FLSYR” and “c(2NapA)LS(2NapA)R” means “cyclic (2NapA)LS (2NapA)R”. The terms “2NapA” and “2-Nal” are abbreviations for 2-naphthylalanine. In one example, the peptide inhibitor is cyclo-((2-Nal)-Leu-Ser-(2-Nal)-Arg) (c2).

In alternative examples, the peptide inhibitor is not cyclo-((2-Nal)-Leu-Ser-(2-Nal)-Arg).

In another example, the inhibitor is protein inhibitor. In another example, the inhibitor is an antibody or fragment thereof. The peptide or protein may be any peptide or protein capable of binding to vimentin and capable of at least partly blocking or occluding the hGIIA binding domain (e.g., capable of binding to coil 2 of vimentin) thereof. The inhibitor may disrupt a direct physical association between vimentin and hGIIA. Without wishing to be bound by any theory, the inventors have found that c2 binds to amino acid residues within the amino acid residues numbered 347 to 357, and 342 to 350 which are located on vimentin coil 2. The inhibitor may bind to any one or more amino acids within coil 2 as defined herein. The inhibitor may bind to residues 347 to 357 of vimentin. The inhibitor may bind to residues 342 to 350 of vimentin. The inhibitor may bind to any one or more of the following residues on vimentin: M347, N350, F341, V353, E354 or N357. The molecular modelling analysis performed herein has indicated that these residues may directly contact an effective vimentin inhibitor. The inhibitor may bind to any one or more of the following residues on vimentin: M347, F341 and E354. The inhibitor may at least bind to residues M347, F341 and E354 on vimentin.

Suitably, the inhibitor is an antigen-binding molecule. In some examples, the antigen-binding molecule is an immunoglobulin. In another example, the inhibitor is an antibody or fragment thereof.

The term “immunoglobulin” will be understood to include any binding agent comprising an immunoglobulin domain. Exemplary immunoglobulins are antibodies. Additional proteins encompassed by the term “immunoglobulin” include domain antibodies, camelid antibodies and antibodies from cartilaginous fish (i.e., immunoglobulin new antigen receptors (IgNARs)). Generally, camelid antibodies and IgNARs comprise a VH domain, however lack a VL domain and are often referred to as heavy chain immunoglobulins.

The skilled artisan will be aware that an “antibody” is generally considered to be a protein that comprises a variable region made up of a plurality of polypeptide chains, e.g., a polypeptide comprising a VL and a polypeptide comprising a VH. An antibody also generally comprises constant domains, some of which can be arranged into a constant region, which includes a constant fragment or fragment crystallizable (Fc), in the case of a heavy chain. A VH and a VL interact to form a Fv comprising an antigen binding region that is capable of specifically binding to one or a few closely related antigens. Generally, a light chain from mammals is either a κ light chain or a λ light chain and a heavy chain from mammals is α, δ, ε, γ, or μ. Antibodies can be of any type (e.g., IgG, IgE, IgM, IgD, IgA, and IgY), class (e.g., IgG1, IgG2, IgG3, IgG4, IgA1 and IgA2) or subclass. The term “antibody” also encompasses humanized antibodies, primatized antibodies, human antibodies and chimeric antibodies.

As used herein, the term “Fv” shall be taken to mean any protein, whether comprised of multiple polypeptides or a single polypeptide, in which a VL and a VH associate and form a complex having an antigen binding site, i.e., capable of specifically binding to an antigen. The VH and the VL which form the antigen binding site can be in a single polypeptide chain or in different polypeptide chains. Furthermore, an Fv of the disclosure (as well as any protein of the disclosure) may have multiple antigen binding sites which may or may not bind the same antigen. This term shall be understood to encompass fragments directly derived from an antibody as well as proteins corresponding to such a fragment produced using recombinant means. In some examples, the VH is not linked to a heavy chain constant domain (CH) 1 and/or the VL is not linked to a light chain constant domain (CL). Exemplary Fv containing polypeptides or proteins include a Fab fragment, a Fab′ fragment, a F(ab′) fragment, a scFv, a diabody, a triabody, a tetrabody or higher order complex, or any of the foregoing linked to a constant region or domain thereof, e.g., CH2 or CH3 domain, e.g., a minibody. A “Fab fragment” consists of a monovalent antigen-binding fragment of an immunoglobulin, and can be produced by digestion of a whole antibody with the enzyme papain, to yield a fragment consisting of an intact light chain and a portion of a heavy chain or can be produced using recombinant means. A “Fab′ fragment” of an antibody can be obtained by treating a whole antibody with pepsin, followed by reduction, to yield a molecule consisting of an intact light chain and a portion of a heavy chain comprising a VH and a single constant domain. Two Fab′ fragments are obtained per antibody treated in this manner. A Fab′ fragment can also be produced by recombinant means. A “F(ab′)2 fragment” of an antibody consists of a dimer of two Fab′ fragments held together by two disulfide bonds, and is obtained by treating a whole antibody molecule with the enzyme pepsin, without subsequent reduction. A “Fab2” fragment is a recombinant fragment comprising two Fab fragments linked using, for example a leucine zipper or a CH3 domain. A “single chain Fv” or “scFv” is a recombinant molecule containing the variable region fragment (Fv) of an antibody in which the variable region of the light chain and the variable region of the heavy chain are covalently linked by a suitable, flexible polypeptide linker.

In some examples, the antibody may be a single domain antibody (sdAb), for example a nanobody. An sdAb is an antibody fragment consisting of a single monomeric variable antibody domain. Like a whole antibody, it is able to bind selectively to a specific antigen. With a molecular weight of only 12-15 kDa, single-domain antibodies are much smaller than common antibodies (150-160 kDa) which are composed of two heavy protein chains and two light chains, and even smaller than Fab fragments (˜50 kDa, one light chain and half a heavy chain) and single-chain variable fragments (˜25 kDa, two variable domains, one from a light and one from a heavy chain).

As used herein, the term “binds” in reference to the interaction of a compound or an antigen binding site thereof with an antigen means that the interaction is dependent upon the presence of a particular structure (e.g., an antigenic determinant or epitope) on the antigen.

As used herein, the term “specifically binds” or “binds specifically” shall be taken to mean that a compound of the disclosure reacts or associates more frequently, more rapidly, with greater duration and/or with greater affinity with a particular antigen or cell expressing same, or epitope, than it does with alternative antigens or cells or epitopes. For example, a compound binds to vimentin with materially greater affinity (e.g., 20 fold or 40 fold or 60 fold or 80 fold to 100 fold or 150 fold or 200 fold) than it does to other receptors or to antigens commonly recognized by polyreactive natural antibodies (i.e., by naturally occurring antibodies known to bind a variety of antigens naturally found in humans). In another example, an inhibitor may bind to coil 2 of vimentin with materially greater affinity (e.g., 20 fold or 40 fold or 60 fold or 80 fold to 100 fold or 150 fold or 200 fold) than it does to other regions of the vimentin protein.

As used herein, the term “antigen binding site” shall be taken to mean a structure formed by a protein that is capable of binding or specifically binding to an antigen. The antigen binding site need not be a series of contiguous amino acids, or even amino acids in a single polypeptide chain. For example, in a Fv produced from two different polypeptide chains the antigen binding site is made up of a series of amino acids of a VL and a VH that interact with the antigen and that are generally, however not always in the one or more of the CDRs in each variable region. In some examples, an antigen binding site is a VH or a VL or a Fv.

As used herein, the term “epitope” (syn. “antigenic determinant”) shall be understood to mean a region of an antigen, for example vimentin, to which a protein comprising an antigen binding site of an antibody binds. This term is not necessarily limited to the specific residues or structure to which the protein makes contact. For example, this term includes the region spanning amino acids contacted by the protein and/or 5-10 or 2-5 or 1-3 amino acids outside of this region. In some examples, the epitope comprises a series of discontinuous amino acids that are positioned close to one another when vimentin is folded, i.e., a “conformational epitope”. The skilled artisan will also be aware that the term “epitope” is not limited to peptides or polypeptides. For example, the term “epitope” includes chemically active surface groupings of molecules such as sugar side chains, phosphoryl side chains, or sulfonyl side chains, and, in certain examples, may have specific three dimensional structural characteristics, and/or specific charge characteristics.

The protein inhibitor may be a mutant vimentin protein that has reduced binding capability to the hGIIA protein or a mutant hGIIA protein that has reduced binding capability to the vimentin protein. Without wishing to be bound by theory, such proteins may act as decoys to the endogenous vimentin and hGIIA proteins, saturating the available binding sites on the endogenous proteins and thereby inhibiting their function.

In another example, the inhibitor may be a small molecule inhibitor.

In light of the present disclosure, the person skilled in the art may conduct screening assays to identify inhibitors or agents, that bind to and/or modulate the activity of vimentin and/or hGIIA. In one example, the binding of the inhibitor or agent to vimentin is assessed through an enzyme immunoassay.

The activity of vimentin can be assessed by standard methods known in the art for assessing cellular vimentin activity.

The effect of the presence of the candidate test compounds on cell growth and/or proliferation (e.g., of cancer cell lines such as prostate cancer cell lines), may also be assessed. For example, the effect of the candidate compounds on xenograft mouse models can be assessed. In another example, the effect of the presence of the candidate test compounds on inflammation may also be assessed. This may be achieved, for example, by determining the level of expression and/or activity of one or more markers of inflammation (e.g., a prostaglandin, cytokine, or any other known marker of inflammation).

Thus, the present disclosure provides methods to identify candidate compounds that may inhibit vimentin activity when administered to a cell, tissue, or subject. Such methods may be carried out in vivo, for example in animal subjects; or using in vitro and/or ex vivo assays, such as described herein.

It is also contemplated that the candidate agent or inhibitor may be rationally designed or engineered de novo based on desired or predicted structural characteristics or features that indicate the candidate agent could block or inhibit binding of Vimentin to hGIIA at coil 2 of vimentin. In other examples, the candidate agent may be identified by screening a library of molecules without initial selection based on desired or predicted structural characteristics or features that indicate the candidate modulator could block or inhibit binding of Vimentin to hGIIA at coil 2 of vimentin. Such libraries may comprise randomly generated or directed libraries of proteins, peptides, nucleic acids, recombinant antibodies or antibody fragments (e.g., phage display libraries), carbohydrates and/or lipids, libraries of naturally-occurring molecules and/or combinatorial libraries of synthetic organic molecules.

Non-limiting examples of techniques applicable to the design and/or screening of candidate agents may employ X-ray crystallography, NMR spectroscopy, computer assisted screening of structural databases, computer-assisted modelling or biochemical or biophysical techniques which detect molecular binding interactions, as are well known in the art.

Biophysical and biochemical techniques which identify molecular interactions include competitive radioligand binding assays, co-immunoprecipitation, fluorescence-based assays including fluorescence resonance energy transfer (FRET) binding assays, electrophysiology, analytical ultracentrifugation, label transfer, chemical cross-linking, mass spectroscopy, microcalorimetry, surface plasmon resonance and optical biosensor-based methods, such as provided in Chapter 20 of CURRENT PROTOCOLS IN PROTEIN SCIENCE Eds. Coligan ei 1., (John Wiley & Sons, 1997) Biochemical techniques such as two-hybrid and phage display screening methods are provided in Chapter 1 of CURRENT PROTOCOLS IN PROTEIN SCIENCE Eds. Coligan et al, (John Wiley & Sons, 1997).

Accordingly, an earlier step of the method may include identifying a plurality of candidate molecules that are selected according to broad structural and/or functional attributes, such as an ability to bind coil 2 of vimentin.

Additionally, the present method may further include one or more of the steps of:
selecting a candidate agent that inhibits or blocks binding of Vimentin to hGIIA at coil 2 of vimentin;
isolating or purifying the candidate agent;
formulating the candidate agent into a pharmaceutical formulation; and
adding the candidate agent or the pharmaceutical formulation to packaging and/or a container, such as a vial, ampoule, bag, blister pack, bottle, cartridge, injection needle, injection syringe, single dose container, strip of multiple single dose containers, or tube.

### Compositions

Compositions comprising a compound that inhibits vimentin activity or a compound that disrupts the vimentin-hGIIA interaction, together with an acceptable carrier or diluent, are useful in the methods disclosed herein. Therapeutic compositions can be prepared by mixing the desired compounds having the appropriate degree of purity with optional pharmaceutically acceptable carriers, excipients, or stabilizers (see, e.g., Remington's Pharmaceutical Sciences, 16th edition, Osol, A. ed. (1980)), in the form of lyophilized formulations, aqueous solutions or aqueous suspensions. Acceptable carriers, excipients, or stabilizers are preferably nontoxic to recipients at the dosages and concentrations employed, and include buffers such as Tris, HEPES, PIPES, phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; sugars such as sucrose, mannitol, trehalose or sorbitol; salt-forming counter-ions such as sodium; and/or non-ionic surfactants such as TWEEN™, PLURONICS™ or polyethylene glycol (PEG).

Additional examples of such carriers include ion exchangers, alumina, aluminum stearate, lecithin, serum proteins, such as human serum albumin, buffer substances such as glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, colloidal silica, magnesium trisilicate, polyvinyl pyrrolidone, and cellulose-based substances.

Therapeutic compositions to be used for in vivo administration should be sterile. This is readily accomplished by filtration through sterile filtration membranes, prior to or following lyophilization and reconstitution. The composition may be stored in a lyophilized form or in solution if to be administered systemically. If in a lyophilized form, it is typically formulated in combination with other ingredients for reconstitution with an appropriate diluent at the time for use. An example of a liquid formulation is a sterile, clear, colourless unpreserved solution filled in a single-dose vial for subcutaneous injection.

Single or multiple administrations of the compositions are administered depending on the dosage and frequency as required and tolerated by the patient. The dosage and frequency will typically vary according to factors specific for each patient depending on the specific therapeutic or prophylactic agents administered, the severity and type of disease or condition, the route of administration, as well as age, body weight, response, and the past medical history of the patient. Suitable regimens can be selected by one skilled in the art by considering such factors and by following, for example, dosages reported in the literature and recommended in the Physician's Desk Reference (56th ed., 2002). Generally, the dose is sufficient to treat or ameliorate symptoms or signs of disease without producing unacceptable toxicity to the patient.

### EXAMPLES

### Example 1. hGIIA-Mediated Tumorigenesis is Blocked by c2

### Murine Prostate Cancer Xenograft Model

Animals (male Balb/c nu/nu mice, aged six weeks, n=15 per group) were injected subcutaneously in the right rear flank with 1×10⁶ LNCaP prostate cancer cells. Tumour size was monitored with calipers measuring tumour width and length and tumour volume calculated as described (Sved et al., Cancer Res, 2004. 64(19): p. 6934-40). When tumours were palpable and measured >5×5 mm, animals were randomised and entered into the study. Animals were treated thrice weekly with either normal saline (vehicle) or cyclo-((2-Nal)-Leu-Ser-(2-Nal)-Arg) (termed c2, 0.1 mg/kg in saline) and tumour volume (calculated as V=p/6 (d1.d2)^{3/2} in mm³) monitored over an eight-week period. Tumour volume data are mean±SE.

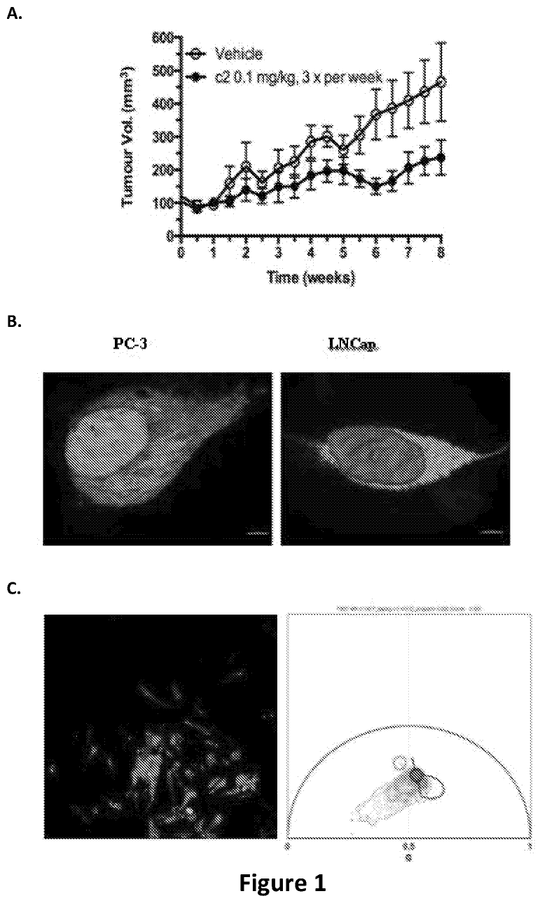

As illustrated in FIG. 1A, c2 significantly reduced the growth in tumour volume in the animals compared to vehicle.

Colocalisation of hGIIA and Vimentin in Prostate Cancer Cells

### Preparation of Antibodies for Immunofluorescence.

Anti-sPLA₂ murine monoclonal antibody 4A1 (Smith, G. M et al., 1992) was diluted to 1 mg/mL in PBS. Alexa Fluor 568 (AF 568) succinimidyl ester (Thermofisher Scientific, Massachusetts, US) were emulsified in 1 mL of DMSO and dried in a vaccum desiccator. 100 μL of 1 mg/mL of 4A1 antibody was added to 10 μL of 1 M NaHCO₃ and then 30 μg of AF 568. The solution was mixed for 4 hours, then added to Bio-spin 30 Tris column (BioRad Laboratories, Hercules, Calif., USA) and centrifuged at 1100×g for 3 minutes to remove unconjugated dye and antibody. The absorbance of the conjugated antibody is measured with Nanodrop 2000c at A₂₈₀ (peak absorbance of protein) and A₅₇₈ (peak absorbance of AF568). The degree of labelling (DOL) was calculated using the following equation:

$$DOL = \frac{{A}_{578}}{\begin{matrix}( extinction coefficient \times \\ ( {A}_{280} - {A}_{578} \times coefficient factor ) ) \div 203000 )\end{matrix}}$$

A DOL >5 was used for immunofluorescent imaging.

Vimentin antibody RV202 conjugated to Alexa Fluor 488 (AF 488) were from BD Biosciences (NJ, USA).

### Cell Culture

Prostate cancer cell lines PC-3, LNCaP, and DU145 were chosen for their range of hGIIA and vimentin expression. PC-3 was grown in RPMI (Lonza, Basel, Switzerland), LNCaP in DMEM (Lonza), and DU145 in MEM (Sigma-Aldrich) cell culture, all with 10% FCS, 4 mM L-glutamine (Sigma-Aldrich) and 1% penicillin and streptomycin. Cells were incubated at 37° C. and 5% CO₂, and sub-cultured when neared confluency. Cells were detached by 0.05% trypsin in PBS for 5 minutes. Trypsin was neutralised with an equal volume of cell media, and cells were pelleted by centrifugation at 50×g for 5 minutes and the cells were reconstituted in the growth medium.

### Immunofluorescence Staining

Cell lines PC-3, LNCaP and DU145 were grown on Millicell 8-well chamber slides (Merck, Darmstadt, Germany) overnight. Cells were washed with phosphate buffer (PB) and then fixed with 250 μL of 4% paraformaldehyde for 10 minutes. 0.2% Triton X-100 was added to cells for 10 minutes, removed then blocked with 5% FCS in PB for 10 minutes. Blocking solution was then removed and cells were stained with 100 μL of three antibodies for one hour; 4A1/AF568 conjugated antibody at a 1:50 dilution from stock, RV202/AF488 (BD Biosciences) at a 1:100 dilution, and α-E-Cadherin/AF647 (BD Biosciences) at a 1:100 dilution. The conjugated antibodies were then removed and cells were washed with 250 μL of PB for 5 minutes. This wash was repeated twice. PB was removed from the wells and the chamber was taken off the slide. Cells were mounted with prolong gold anti-fade with DAPI (Thermo Fisher Scientific, Waltham, Ma, USA) coverslip was added and slide sealed with nail polish.

### Confocal Imaging

Slides were imaged with a Zeiss LSM 800 confocal microscope (Zeiss, Oberkochen, Germany) with a 63× oil immersion objective. Sequential scanning was used to remove bleed-through or crosstalk between the channels. Samples were excited with 405 nm, 488 nm and 561 nm lasers to excite DAPI, AF488/α-vimentin and AF568/α-hGIIA respectively and image acquired with airyscan detector. Z-stacks were also imaged, using 0.2 μm step size.

As illustrated in FIG. 1B (left image, PC-3 cells; right image, LNCaP cells), hGIIA colocalises with vimentin in prostate cancer cell lines (red, vimentin immunofluorescence; green, hGIIA immunofluorescence; yellow, colocalisation).

### Lifetime Imaging

PC-3 and LNCaP cells were fixed, permeabilized, blocked and stained as above with α-vimentin/AF488 and α-hGIIA/AF568, as AF488 and AF568 act as a FRET donor-acceptor pair. Samples were imaged with a Zeiss LSM 880 (Zeiss, Oberkochen, Germany), and samples excited with 2-photon excitation tuned to 960 nm at 80 MhZ. The fluorescence lifetime of the donor was captured, and collected with the 520-535 nm filter. Lifetime Fluorescence lifetime was processed using the PicoQuant system and captured until fluorescence lifetime has decayed to background levels. For calibration, the lifetime of cells stained with only donor fluorescence (α-vimentin/AF488) is captured (0% FRET point), as well as unstained cells (100% FRET point). Fluorescence lifetime was then captured in cells that are stained with both α-vimentin/AF488 and α-hGIIA/AF568, and had been incubated with and without c2 at 75 μg/mL. All captured fluorescence lifetime is then analysed using Globals SimFCS software for FLIM (Laboratory for Fluorescence Dynamics, University of California, Irvine), which transforms fluorescence decay curves to a phasor plot. Samples were referenced against Atto 488 which has a known lifetime of 4.18 ns, and FRET efficiency calculated.

This imaging study demonstrates, for the first time, that hGIIA binds directly to vimentin. As illustrated in FIG. 1C (left image), Forster resonance energy transfer (FRET) analysis of LNCaP cells revealed pink areas confirming direct binding resonances between vimentin and hGIIA, captured using Fluorescence lifetime imaging microscopy (FLIM). As described above, vimentin (donor) and hGIIA (acceptor) were imaged using antibodies conjugated to Alexa fluor 488 and Alexa fluor 568, respectively. FIG. 1C (right image) shows a phasor plot of fluorescent lifetimes used to calculate FRET efficiency.

### Example 2. Coil 2 of Vimentin Binds Directly to hGIIA In Vitro

Optimisation of Human Vimentin Gene for Recombinant Production in Escherichia coli and Production of Expression Vector

The wild type human vimentin (VIM) mRNA sequence was acquired from the National Center for Biotechnology Information (NCBI) Reference Sequence (RefSeq) accession NM_003380.3. The acquired sequence was modified for optimal incorporation into a heterologous E. coli expression system using the method adapted from Sun et al. (Sun, G. et al. 2016) The method involved the use of GeneOptimizer software (Raab, D. et al., 2010) for a primary optimisation. Any codon that gave more than five adenines in succession in the produced sequence was manually adjusted appropriately to prevent premature polyadenylation. The sequence was inspected by scanning for any restriction site sequences that are utilised in the subsequent subcloning step, namely BamHI (GGATCC) and NdeI (CATATG). The final process of the inspection was to translate the DNA sequence to protein sequence and to compare it with the wild type protein sequence (RefSeq accession NP_003371.2) using Basic local alignment search tool (BLAST) (Altshul et al., 1990) to confirm the identity.

The gene synthesis and the subsequent subcloning into the expression vector were all conducted by GenScript (Piscataway N.J., USA). There were three vimentin expression vectors produced for expression of three different segments of the protein. The entire optimised sequence was used as the open reading frame (ORF) for the expression vector of the full-length vimentin. The DNA sequence comprising the protein residues 96 to 253 was used as ORF for the coil 1 segment and the sequence comprising the residues 254 to 405 was used for the coil 2 segment. On each of the ORF sequences, further modifications were made to ensure correct subcloning and minimise any potential mistranslations. An NdeI restriction site was added to the 5′ end of the ORF, while the 3′ end was modified to include double stop codons (TAATAA), followed by a BamHI restriction site, so that the final sequence has a format of NdeI-ORF-Stop-Stop-BamHI. The synthesised gene was firstly subcloned into a pUC57 plasmid via NdeI and BamHI sites and then ligated into a pET15b expression vector using the same restriction sites. The pET15b vector adds a histidine tag (HHHHHH) and a thrombin cleavage site (LVPRGS) to the N-terminus of the expressed protein. The lyophilised plasmids were reconstituted to 40 μg/mL concentrations by addition of ultrapure water and stored at −20° C.

### Transformation of Vimentin Expression Vector

Rosetta 2 E. coli competent cells were thawed on ice and combined with KCM buffer (100 mM KCl, 30 mM CaCl₂), 50 mM MgCl₂) in a 1:1 ratio to make up 50 μL of the mixture per vial. Two vials of the mixtures were prepared and 2 μL of the expression vector plasmid solution was mixed into one vial. The mixture without the plasmid served as a control. Both mixtures were incubated on ice for 5 minutes. Heat shock was performed by placing them into a water bath set at 42° C. for 90 seconds. Each of the mixtures were immediately added with 100 μL of autoclaved Luria-Bertani (LB) broth and incubated in a water bath at 37° C. for 30 minutes. Each of the mixtures was spread on a 100 mm plate of LB-agar containing ampicillin (100 μg/mL) and chloramphenicol (25 μg/mL). Both plates were incubated overnight at 37° C. The two plates were inspected and if the plate with the plasmid added developed bacterial colonies and the control plate was clear of any colonies, the transformation was determined to be successful. The plates with a successful transformation were sealed and stored for up to 14 days at 4° C.

### Bacterial Amplification and Induction of the Protein.

One to two successfully transformed bacterial colonies were inoculated to 100-200 mL of autoclaved LB broth with 100 μg/mL ampicillin and placed in a shaking incubator at 37° C. overnight. The OD₆₀₀ of the bacterial culture was measured and diluted in up to 3 L of autoclaved LB broth with 100 μg/mL ampicillin to make the final OD₆₀₀ value 0.1. The prepared culture was incubated at 37° C. under agitation, until the OD₆₀₀ value was between 0.6 and 0.8, where the bacteria reach the middle of the exponential growth phase. The growth time typically took 90 to 180 minutes and it was necessary to check the OD₆₀₀ every 10 minutes in the specified period.

Once the OD₆₀₀ value was within the desired range, the induction was initiated by the addition of isopropyl β-D-1-thiogalactopyranoside (IPTG) at 1 mM concentration and further incubated for 120 to 180 minutes under agitation at 37° C. The incubation was stopped before the OD₆₀₀ exceeded 2.5. Successful induction was confirmed through sodium dodecyl sulfate polyacrylamide gel electrophoresis (SDS-PAGE) analysis by comparing the pre- and post-induction cultures.

The bacterial cell suspension was centrifuged at 11000×g for 30 minutes at 4° C. The supernatant was discarded and the cell pellet was resuspended in phosphate buffered saline (PBS). The cell suspension was centrifuged again for a further 30 minutes at the same conditions and the pellet was stored at −20° C. for long-term storage until further purification was performed.

### Purification of Vimentin Protein in Non-Denaturing Conditions

Coil 1 and coil 2 fragments of vimentin were highly soluble in most aqueous buffers. Therefore, these proteins were suitable to be purified under non-denaturing conditions. The components of the buffers used during purification of vimentin are shown in Table 1. In both methods of elution, the His-tagged protein was eluted by exploiting the differences in pH and concentration gradient of imidazole from immobilised metal affinity chromatography (IMAC).

TABLE 1
Components of buffers used during purification of recombinant
His-tagged vimentin proteins

| | Vimentin coil 1 | Vimentin coil 2 | |
|---|---|---|---|
| | fragment | fragment | Full length vimentin |
| Binding buffer / Lysis | 20 mM imidazole, PBS | 20 mM Tris-HCl at | 100 mM Monosodium |
| buffer | 2× | pH 8.0, 200 mM NaCl, | phosphate, 10 mM Tris, |
| | | 20 mM imidazole | 8M urea, adjusted to |
| | | | pH 8.0 with NaOH |
| Wash buffer | 40 mM imidazole, PBS | 20 mM Tris-HCl at | 100 mM Monosodium |
| | 2× | pH 8.0, 200 mM NaCl, | phosphate, 10 mM Tris, |
| | | 50 mM imidazole | 8M urea, adjusted to |
| | | | pH 6.3 with HCl |
| Elution buffer | 200 mM imidazole, | 20 mM Tris-HCl at | 100 mM Monosodium |
| | PBS 2× | pH 8.0, 200 mM NaCl, | phosphate, 10 mM Tris, |
| | | 200 mM imidazole | 8M urea, adjusted to |
| | | | pH 4.5 with HC1 |

The frozen bacterial cell pellet was thawed on ice and resuspended in 20 to 50 mL of binding buffer. Lysozyme was added at 1 mM concentration and incubated on ice for 20 minutes. The cells were lysed through sonication using a Branson Sonifier 250 (Emerson Electronic, Ferguson, Mo., USA) equipped with a microtip, for 30 seconds per cycle, and allowing a sufficient cool down period between the cycles until the lysate was homogeneous with no visible solid particles. The lysate was centrifuged at 11000×g for 30 minutes at 4° C., and the supernatant was collected as the soluble fraction while the sediment was resuspended in PBS as the insoluble fraction. The presence of the target protein in the soluble fraction was confirmed by SDS-PAGE.

A nickel-nitrilotriacetic acid (Ni-NTA) agarose (Qiagen, Hilden, Germany) slurry (binding capacity of 50 mg protein per mL) was loaded at a volume of 3 mL in a glass Econo-Column® chromatography column (Bio-Rad Laboratories, Hercules, Calif., USA) and washed with at least five column volumes of the binding buffer three times. The isolated soluble fraction of the lysate was loaded onto the column and incubated on a rotating mixer for 30 minutes at 4° C. Once the beads had settled to the bottom of the column, the flow through fraction was collected. The beads were washed with at least three column volumes of wash buffer three times and the wash was collected. This was followed by the elution of the His-tagged protein with 3-5 mL of elution buffer. The buffer was allowed to equilibrate with the agarose beads for 5 minutes before the collection of the eluate was made. The elution cycle was repeated 3 times. The purity and yield of the purification was analysed by testing the collected samples by SDS-PAGE, protein assay, infrared spectroscopy and mass spectrometry. The purified proteins were aliquoted and stored at −80° C. until use.

### Purification of Vimentin Protein in Denaturing Conditions

The full-length vimentin was only sparingly soluble in aqueous buffers. Therefore, it was purified under denaturing conditions in the presence of urea (8 M). The overall process of the purification in denaturing conditions is nearly identical to the purification under the non-denaturing conditions outlined above, but with the following differences:
1) Urea (8 M) was added to all buffers used in the purification process.
2) The centrifugation after the sonication was not performed as the separation of the soluble and insoluble fractions was not necessary.
3) It was possible for urea to crystallise at a low temperature. After the mixing step at 4° C., the mixture was inspected for solid crystals. If any were discovered, they were redissolved at a room temperature.

### Purification of 10B2 Antibody

Mouse ascites containing 9.5 mg/mL of anti-hGIIA monoclonal 10B2 antibody (Smith et al. 1992) (Bioquest, Sydney, Australia) was filtered through syringe filters with a low protein binding membrane, either using Millex® polyvinyl fluoride (PVDF) 0.22 μm (Merck Millipore, Billerica, Mass., USA) or Corning® surfactant-free cellulose acetate (SFCA) 0.22 μm devices (Corning Inc., Corning, N.Y., USA), in order to minimise clogging of the column.

An ÄKTA purifier 10 (GE Healthcare, Chicago, Ill., USA) fast protein liquid chromatography (FPLC) system was used, which was equipped with an 884 μL loading loop. A 1 mL HiTrap™ Protein G HP affinity column (GE Healthcare) was installed with the ÄKTA system. The column was washed with at least ten column volumes of 20% ethanol in water at flow rate of 3 mL/min, followed by at least ten column volumes of water. The column was then equilibrated with at least ten column volumes of binding buffer (20 mM sodium phosphate, pH 7.0) and the flow rate changed to 1 mL/min at this step. For each cycle of chromatography, the loop was loaded with 440 μL of ascites, and the loop was emptied with five column volumes of binding buffer to elute all unbound proteins from the column. Once the UV₂₈₀ reading stabilised to the baseline level, elution buffer (0.1 M glycine HCl, pH 2.7) was loaded onto the column and a sharp UV₂₈₀ peak that corresponds to pure 10B2 was observed within six column volumes. The acidic eluate was neutralised by adding 10% volume of 1 M Tris base, before storage at 4° C. The column was re-equilibrated with ten column volumes of binding buffer before loading filtered ascites to the loop again. For storage, the column was firstly washed with at least ten column volumes of water followed by ten column volumes 20% ethanol in water at 3 mL/min flow rate, then stored at 4° C. The buffer containing the purified antibody was exchanged to 0.5 M sodium phosphate, pH 7.5, using a Vivaspin® 20 (GE Healthcare) centrifugal concentrator.

### Coupling of 10B2 Antibody to Sepharose

Purified 10B2 was coupled to CNBr-activated Sepharose 4 Fast Flow beads (Sigma-Aldrich, St. Louis, Mo., USA) according to the manufacturer's instructions. The buffer containing the 10B2 antibody was changed to coupling buffer (100 mM sodium bicarbonate, pH 8.3, 500 mM NaCl) with a Vivaspin® 20 centrifugal concentrator (Sigma-Aldrich, Sydney, NSW, Australia). Lyophilised CNBr-activated Sepharose 4 Fast Flow beads were swelled and activated by six rounds of soaking 1.5 g of beads with 5 mL of 1 mM HCl solution for 5 minutes at 4° C. in a 50 mL centrifuge tube, followed by centrifuging at 200×g for 1 minute and removal of the supernatant. Purified antibody (10 mg/mL, 5 mL) was added to the sedimented beads and incubated at room temperature on a rolling mixer for 2 hours. The excess antibody solution was removed and the beads were washed three times with 10 mL of coupling buffer for 5 minutes. Unbound beads were blocked by adding 10 mL of 100 mM Tris-HCl (pH 8.0) buffer and mixing on a rolling mixer for 2 hours at room temperature. The beads were washed with 10 mL of 100 mM sodium acetate (pH 4.0), 500 mM NaCl buffer three times. The beads were washed again three times with 10 mL of 100 mM Tris-HCl (pH 8.0), 500 mM NaCl buffer. The beads were washed again with excess 20% ethanol in water. The coupled beads were stored at 4° C. in 20% ethanol in water.

hGIIA Purification Using Affinity Chromatography

Affinity-based purification of hGIIA was conducted using a method adapted from Tseng et al. (Tseng, et al. 1996 J. Biol. Chem. 271, 23992-23998). Frozen conditioned media obtained by fermentation of CHO cell line 2B1 expressing recombinant hGIIA (Edwin Huang, Australian Institute of Bioengineering and Nanotechnology, University of Queensland) was thawed at 4° C. To every 1 L of the media, 49.67 g of NaCl was added to adjust the concentration to 1 M. The media was filtered with a Stericup® vacuum filtration unit through 0.22 μm polyethersulfone (PES) membrane (Merck Millipore, Bayswater, VIC, Australia).

Sepharose 4 Fast Flow coupled with 10B2 antibody (˜3 mL with more than 40 mg of 10B2 bound) was packed into an empty XK16 column (GE Healthcare) as per manufacturer's instructions. The prepared column was installed to the ÄKTA Purifier FPLC chromatography system and washed with 20% ethanol in water, followed by ultrapure water, both for more than five column volumes or until stable zero conductance level was observed, at a flow rate of 3.5 mL/min. The column was equilibrated with more than five column volumes of binding buffer (20 mM sodium phosphate (pH 7.0), 1 M NaCl) at a flow rate of 1 mL/min, until a stable conductance level was established. The sample was loaded to the column and washed with six column volumes of the binding buffer to remove any unbound proteins from the column. Pure hGIIA was eluted with 0.1 M glycine HCl (pH 2.7) elution buffer. The acidic eluate was immediately neutralised by adding 10% volume of 1 M Tris base and stored at 4° C. for short-term prior to buffer exchange. The column was washed with at least five column volumes of ultrapure water and five column volumes 20% ethanol in water at less than 4 mL/min flow rate, then stored at 4° C.

### Protein Analysis by SDS-PAGE

Diluted or undiluted protein sample was mixed with Laemmli Sample Buffer 2× (Bio-Rad Laboratories) in 2:1 ratio with or without 5% 2-mercaptoethanol. The combined sample was heated on a heat block at 90° C. for 10 minutes. The prepared samples, as well as 5 μL of Precision Plus Protein™ Dual Color Standards (Bio-Rad Laboratories), were loaded onto each lane of 12% polyacrylamide TGX stain-free pre-cast gel (Bio-Rad Laboratories). Electrophoresis was performed in running buffer (25 mM Tris, 192 mM glycine, 0.1% SDS, pH 8.3) for 25 minutes at 250 volts.

Gels were stained in Coomassie Blue staining solution (0.1% Coomassie Brilliant Blue G250 (Bio-Rad Laboratories) in 50% methanol) for 40 minutes at room temperature under agitation. Gels were destained in 100 mL of destaining solution (40% methanol, 1% 1 M acetic acid). Typically, clear bands were observed after destaining for 30 minutes under agitation at room temperature. However, up to overnight destaining in non-agitating condition was also acceptable. Destained gels were rinsed with water and digitally imaged with Gel Doc™ EZ System (Bio-Rad Laboratories).

hGIIA ELISA

Quantification of hGIIA was conducted with the sandwich enzyme linked immunosorbent assay (ELISA) approach using a similar method described by Smith et al. (Smith et al. Br J Rheumatol 1992; 31:175-8) A 96-well microplate (Sarstedt, Nümbrecht, Germany) was coated with the hGIIA specific 9C1 monoclonal antibody (Bioquest) of 2 μg/mL concentration in PBS at 100 μL/well overnight at 4° C. Plates were emptied and blocked with 1% bovine serum albumin (BSA) in PBS (200 μL) at 4° C. for at least 16 hours and used within two weeks. Seven hGIIA standards in the range of 1.5625-100 ng/mL were prepared in 0.1% BSA in PBS from a 100 ng/mL stock solution by step dilutions. The hGIIA samples were also diluted by 103 to 10⁶ times in 0.1% BSA in PBS. The blocked plates were washed twice with PBST (PBS with 0.05% Tween-20) at 200 μL/well volume, and hGIIA standards, a blank (0.1% BSA in PBS) and samples were added to the plate at 100 μL/well volume in triplicates. Plates were incubated for 2 hours at 37° C. A secondary 4A1 monoclonal antibody-alkaline phosphatase (AP) conjugate was diluted in 0.1% BSA in PBS to 1:1000. Plates were emptied, washed twice with PBST, and then the prepared 4A1-AP conjugate was added at 100 μL/well and then incubated for 1 hour at 37° C. Solid PNPP (p-nitro-phenyl phosphate) AP substrate (Sigma-Aldrich) was dissolved in carbonate buffer (8 mM Na₂CO₃, 36 mM NaHCO₃, 2 mM MgCl₂, pH 9.8) to 1 mg/mL concentration. Plates were emptied and washed three times with PBST (200 μL/well) and twice with carbonate buffer (200 μL/well). The prepared AP substrate was added at 100 μL/well. Plates were incubated at 37° C. for up to 60 minutes and the absorbance was read at 405 nm by Victor X4 (PerkinElmer, Waltham, Mass., USA) plate reader at 15-minute intervals. The standard curve was plotted as a non-linear regression four parameter logistic (4-PL) sigmoidal curve to calculate the original concentration of the protein. The 4-PL sigmoidal curve is as follows:

$$y = d + \frac{a - d}{1 + {( \frac{x}{C} )}^{b}}$$

### Protein Quantitation by Detergent Compatible Protein Assay

Purified vimentin proteins were quantified using a detergent compatible (DC) protein assay kit (Bio-Rad Laboratories) as per the manufacturer's instructions. BSA standards ranging from 0.125-2 μg/μL were prepared in PBS. Protein samples were diluted in PBS to concentrations within the detectable range. Triplicates of BSA standards, blank (PBS) and diluted protein samples were added to a 96-well microplate (Sarstedt, Newton, N.C., USA), in 5 μL/well volume. The reagents A and S from the kit were combined in a 50:1 ratio, and 25 μL of this was added to each well, and then 200 μL of the reagent B was added to each well. The plate was mixed by gentle agitation, covered in aluminium foil and incubated at room temperature for 15 minutes. The absorbance was measured at 750 nm with either a Spectramax M5e (Molecular Devices, Sunnyvale, Calif., USA) or CLARIOstar (BMG LABTECH, Offenburg, Germany) plate reader. The original protein concentration was calculated from the linear regression of the standard curve plotted.

### Protein Quantitation by Infrared Spectroscopy

As an alternative method to the hGIIA ELISA or DC protein assay, it was possible to quantify the proteins by a Direct Detect infrared spectrometer (Merck Millipore), as per standard protocol. In case of hGIIA quantification, the infrared spectroscopy method consistently provided similar results to the ELISA method with no more than a 10% difference.

### Mass Spectrometry

The mass of vimentin coil 1 and 2 fragments and hGIIA proteins were identified with MALDI-TOF MS. Purified protein samples were desalted by dialysis in grade water using a Pur-A-Lyzer™ Maxi kit (Sigma Aldrich) or exchanging the buffer to grade water using a Vivaspin® 500 (GE Healthcare) centrifugal concentrator. The concentration of the desalted samples were analysed with the DC protein assay (Section 0) and adjusted to 100 μg/mL concentration. Matrices were prepared on the stainless steel plate firstly by placing ethanol saturated with sinapinic acid as the bottom layer. The prepared protein sample in water was mixed in a 1:1 ratio with TA30 solution (30% acetonitrile, 0.1% trifluoroacetic acid in water saturated with sinapinic acid) and placed on the plate as the top layer. Once the matrices were dry, the plate was analysed a Bruker Autoflex speed LRF MALDI-TOF MS system (Bruker, Billerica, Mass., USA) that was calibrated with Protein Standard II (Bruker).

The effect of inhibitors on the hGIIA-vimentin interaction was measured with a colorimetric enzyme immunoassay (EIA), using a similar protocol to the hGIIA ELISA method. Purified vimentin protein solutions (full length, coil 1 or coil 2 fragments) were diluted to 10-20 μg/mL concentration in PBS and seeded to a 96-well microplate with a 100 μL/well volume. After an overnight incubation at 4° C., the plate was emptied. Each well was then blocked with 200 μL of 1% BSA in PBS. The blocked plate was incubated for at least an overnight period at 4° C. and used within 1 week.

The hGIIA solution was prepared with or without inhibitors. For testing the hGIIA binding to vimentin, samples of the hGIIA solution were prepared in a range of 5-1000 ng/mL in PBS with 0.1% BSA, without any inhibitors. The PBS buffer with 0.1% BSA without any hGIIA served as a control. For testing the efficacy of the inhibitors, stock solutions of the inhibitors prepared in DMSO were step diluted in DMSO and added to the solution of purified hGIIA (250 ng/mL, in PBS with 0.1% BSA) to the final concentrations between 5-100 μM. The hGIIA solution added with DMSO (0.4%) but without any inhibitor was prepared as a control. The prepared mixtures were incubated at room temperature for 30 minutes. The final DMSO concentration in all samples did not exceed 0.4%. Two inhibitors tested were c2 and LY311727. LY311727, or [3-[[3-(2-amino-2-oxoethyl)-2-ethyl-1-(phenylmethyl)-1H-indol-5-yl]oxy]propyl]-phosphonic acid; CAS number 164083-84-5, molecular formula C₂₂H₂₇N₂O₅P:

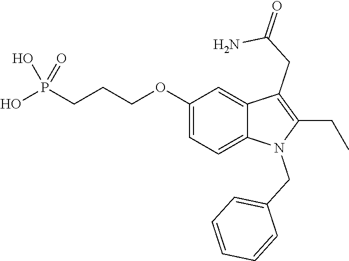 [SMILES: CCc1c(CC(N)=O)c2cc(OCCCP(=O)(O)O)ccc2n1Cc1ccccc1]

is commercially available.

The blocked plate was emptied and washed with 200 μL/well of PBST twice. A 100 μL volume of each prepared hGIIA-inhibitor mixture was added to wells in triplicate and incubated at 37° C. for 1-2 hours. 4A1-AP conjugate was diluted in 0.1% BSA in PBS to 1:1000. Plates were emptied, washed twice with PBST, and then the prepared 4A1-AP conjugate was added (100 μL/well) and then incubated for 1 hour at 37° C.

The AP substrate PNPP powder was dissolved in carbonate buffer to 1 mg/mL concentration. Plates were emptied and washed three times with 200 μL/well volume of PBST, followed by washing twice with carbonate buffer, also at 200 μL/well volume. The prepared PNPP solution was added (100 μL/well) and the plates were incubated at 37° C. for up to 60 minutes and the absorbance at 405 nm was read by a Victor X4 plate reader at 15-minute intervals.

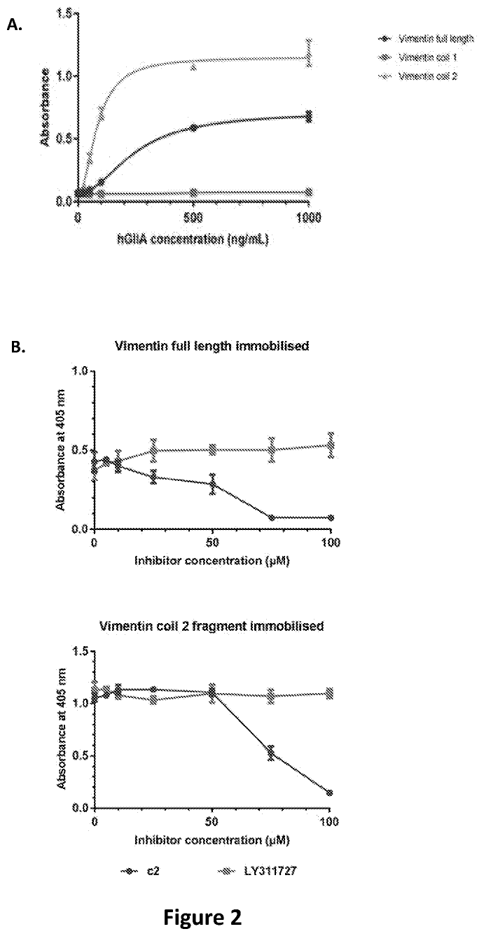

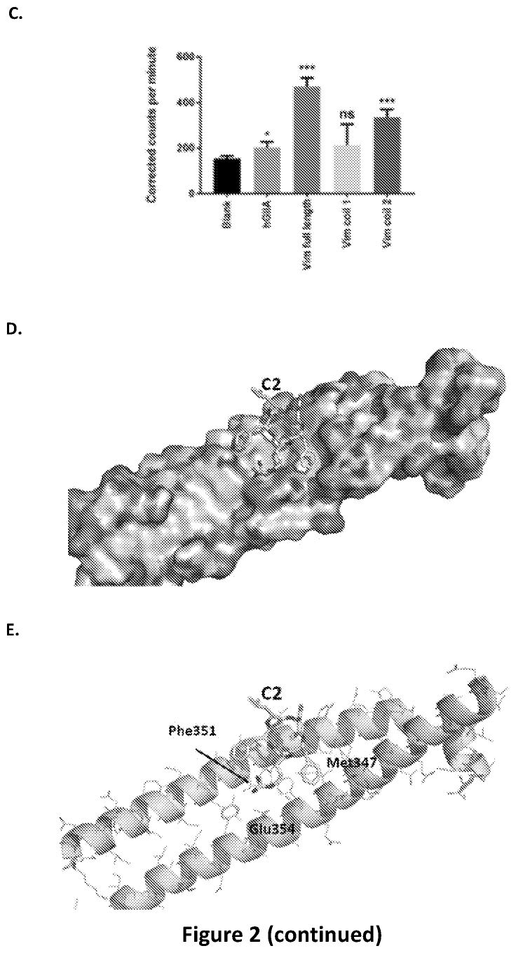

As illustrated in FIG. 2A, hGIIA bound strongly to immobilised vimentin coil 2, significantly less strongly to immobilised full length vimentin, and showed no detectable binding to immobilised vimentin coil 1. This experimental evidence provides further demonstration of the finding described herein, that hGIIA binds specifically to coil 2 of vimentin.

FIG. 2B top panel illustrates the results of incubating full length vimentin with hGIIA in the presence of c2 (blue) or the hGIIA inhibitor LY311727 (red). FIG. 2B bottom panel illustrates the results of incubating coil 2 of vimentin in the presence of c2 (blue) or the hGIIA inhibitor LY311727 (red). As shown, c2 exhibited greater inhibition of hGIIA binding to full length vimentin than LY311727.

### Affinity of Radiolabelled c2 to Immobilised Proteins

To an Isoplate 96-well microplate with white wall and flat clear bottoms (PerkinElmer), 100 μL of either hGIIA, vimentin full length, coil 1 or 2 prepared in PBS to 40 μg/mL concentration or PBS without any proteins, was added, each in triplicate wells. The plate was covered with a lid and incubated at 4° C. overnight. The plate was emptied and blocked by adding 200 μL of 1% BSA in PBS into each well. The plated was covered with a lid and incubated at 4° C. for at least overnight.

The plate was emptied and washed with 200 μL/well of PBST (containing 0.05% Tween-20) five times. The 2 mg/mL stock solution of [3H]c2 compound (SibTech, Brookfield, Conn., USA) was diluted to 50 μM in PBS and 30 μL of the prepared solution was added to each well. The plate was sealed and incubated at 37° C. for 60 min. The plate was emptied and washed with PBST five times. Ultima Gold XR scintillation fluid (PerkinElmer, Waltham, Mass., USA) was added to the washed plate (100 μL/well). The plate was sealed with a clear film and measured using a Microbeta2 scintillation counter (PerkinElmer), with default settings optimised for 3H detection.

As shown in FIG. 2C, 3H-labelled c2 binds weakly to hGIIA and more strongly to full-length and coil 2 of vimentin, but not to coil 1 in the absence of hGIIA. This provides further demonstration of c2's binding to coil 2 of vimentin. Taken together, the results disclosed herein demonstrate that hGIIA binds specifically to coil 2 of vimentin and not to coil 1, the cyclic peptide inhibitor c2 binds specifically to coil 2 of vimentin and not to coil 1, and that c2 inhibits the binding of vimentin to hGIIA. Further, c2's inhibitory effect is greater than other known inhibitors of vimentin, indicating the importance of the specific binding site of c2 on coil 2 of vimentin in achieving its improved inhibitory effect.

### Docking Studies of c2 to Vimentin.

### Docking Method 1 Used in Modelling Shown in FIGS. 2D and 2E

Identification of Binding Region of Vimentin that hGIIA Interacts with

We first used the crystal structure of hGIIA (PDB 3u8h) as the target and performed a “Multiple Copy Simultaneous Search” calculations (Miranker et al, Nature. 1991 Feb. 14; 349(6310):633-6; Zeng, J. Comb Chem High Throughput Screen 2000 Oct. 3(5):355-62) of functional groups on the surface of hGIIA. The chemical functional groups correspond to the side chains of amino acids. Afterwards, sequence pattern was derived from the MCSS minima and we used this pattern to search over the sequence of coil 2 fragment of vimentin. As a result, a sequence region of 342-350 (seq: rqmremeen) was identified as the binding region of hGIIA and as the target region where inhibitor C2 binds.

Modelling c2 and its Derivative c2-Like to the Vimentin Coil 2 Fragment Dimer

The MCSS calculation was performed for c2 on the binding region (residues 342-350) of the vimentin dimer (PDB lgk4). The c2 minima with the strongest binding energy to vimentin dimer was dissolved in water, followed by 10 ns MD simulations. The final structure is shown in FIGS. 2D and 2E

Software quCBit (https://www.medchemsoft.com/) was used for the docking studies.

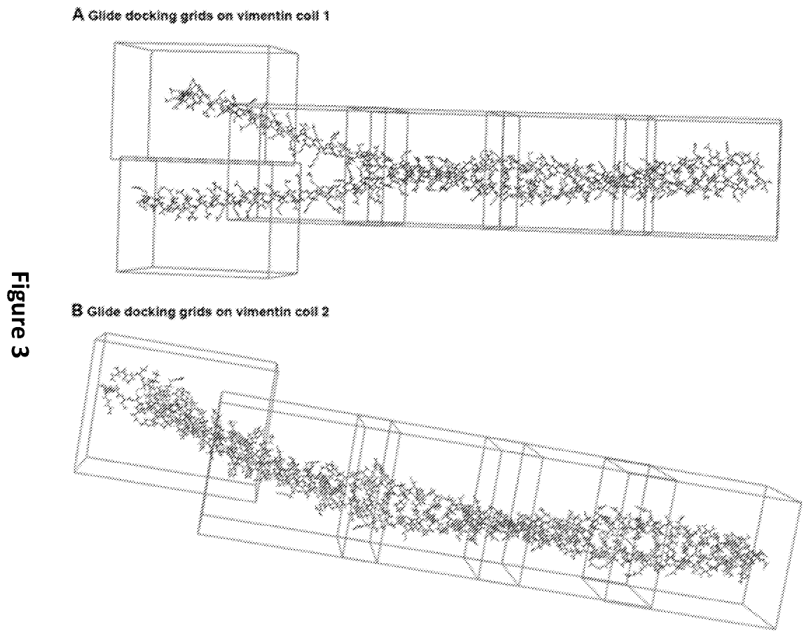

### Docking Method 2 Used in Modelling Shown in FIG. 3

### Preparation of Molecules and Parameterisation

The vimentin coil 1 model was built by aligning the chain A and B of the 3S4R and 3UF1 structures downloaded from the PDB database (Berman et al., 2000) that comprise the 102nd to 249th residues of vimentin in the coiled-coil dimer form. Similarly, the vimentin coil 2 model that comprises the 261st to 406th residues was built from the 3TRT and 1GK4 structures (PDB database). The non-standard 269th and 328th residues found in the 3TRT structure were substituted with the native leucine and cysteine residues respectively.

### Detailed Docking Studies to Coil1 and Coil 2

Docking on vimentin was conducted in individual calculations on the coil 1 and 2 domains of the protein. The docking on vimentin was performed on multiple grids for the inclusion of the entire surface area of each domain of vimentin, as depicted in FIG. 3A. c2 ligand structures were processed by the LigPrep module in Maestro as per default settings using the OPLS 2005 force field (Banks et al., 2005) and were ionized by generating possible states at pH 7.4 using the Epik module (Shelley et al., 2007; Greenwood et al., 2010) (Schrödinger, LLC, New York, N.Y., USA). The generated ligands were docked with Glide XP also using the flexible ligand option to allow for all possible torsional variations.

### Data Analysis and Presentation

The ‘Protein interfaces, surfaces and assemblies’ service at the European Bioinformatics Institute (PISA, http://www.ebi.ac.uk/pdbe/protint/pistart.html) (Krisnel et al., 2007) was used for analysing the interfaces between a protein chain with another protein chain, a ligand or a complex of a protein chain and a ligand. The area of interfaces involved, the solvation free energy gain and the residues involved in the formation of interface were determined in the analysis. The MolProbity server (http://molprobity.biochem.duke.edu/index.php) (Davis et al., 2007) was used to assess the quality of the protein and peptide structures by inspecting aspects such as the overall clash score, Ramachandran plot, rotamers, bond angles and bond lengths. Figures that contain three-dimensional presentation of protein and small molecule structures were generated using PyMOL 1.6 (Delano, 2002) and Maestro software suite 10.2 (Schrödinger, LLC, New York, N.Y., USA).

As illustrated in FIG. 3A, docking was conducted on multiple grids (magenta) to include the entire protein surface area of vimentin coil 1 (A) and 2 (B). The results of this docking analysis revealed that c2 binds to coil 2 of vimentin.

FIG. 3B shows the molecular model of the c2 binding site on vimentin coil 2. The amino acid residues representing sites of binding to the inhibitor c2 are illustrated, based on molecular modelling studies.

Summary of Vimentin Docking Studies with c2.

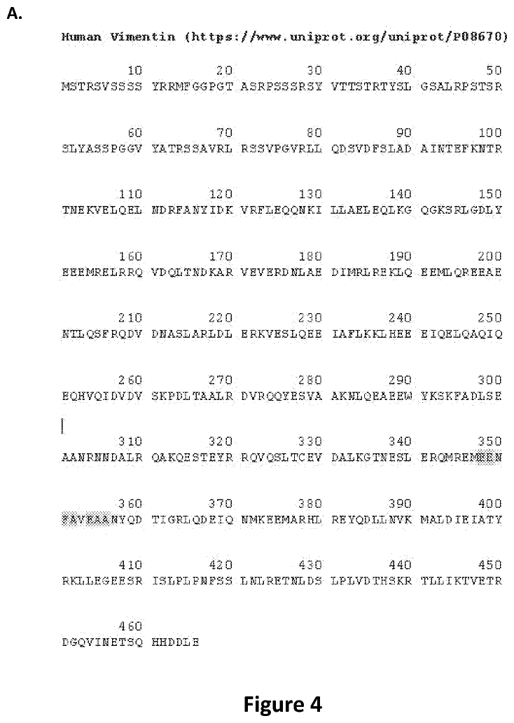

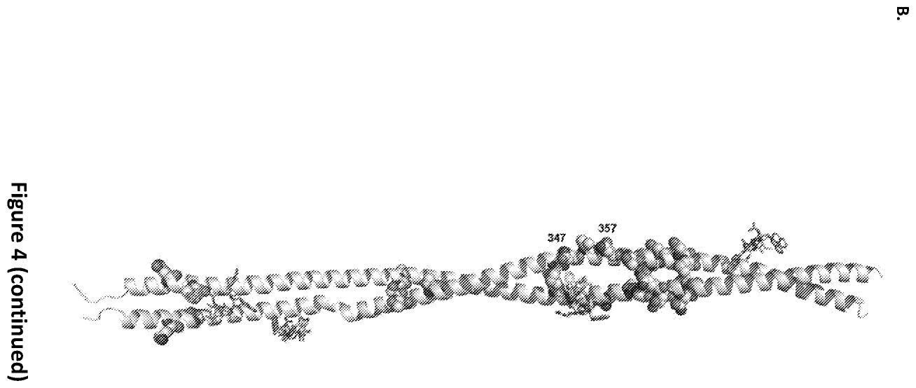

The amino acids that contact c2 in coil 2 of vimentin using both modelling approaches cluster to the same region of coil 2 (viz. within the region 347-357) shown in FIG. 4. Both approaches identify Met 347 as a contact residue, while methodology 1 (indicated by underscored and bolded residues) also identifies Phe 351 and Glu 354 as important in the interaction. Methodology 2 (indicated by overscored bolded residues) additionally identifies Asn 357 as important in the interaction.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

All publications, computer programs, algorithms, protein and nucleic acid sequences (i.e., accession numbers) discussed and/or referenced herein are incorporated herein in their entirety.

Any discussion of documents, acts, materials, devices, articles, or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

1. A method of inhibiting vimentin and/or hGIIA, the method comprising inhibiting the binding of vimentin to hGIIA at coil 2 of vimentin.

2. The method of claim 1, comprising administering an inhibitor of the binding of vimentin to hGIIA at coil 2 of vimentin.

3. The method of claim 2, wherein the inhibitor binds to one or more amino acids within amino acid residues 347 to 357 of vimentin according to the amino acid numbering set out in SEQ ID NO: 3.

4. The method of claim 3, wherein the inhibitor binds to one or more of the following amino acids of vimentin according to the amino acid numbering set out in SEQ ID NO: 3: M347, N350, F351, V353, E354 or N357.

5. The method of any preceding claim, which is a method of inhibiting inflammation.

6. The method of any preceding claim, which is a method of inhibiting epithelial-to-mesenchymal transition.

7. The method of any preceding claim, which is a method of treating or preventing a disease, disorder or condition involving a vimentin-mediated pathway and/or a hGIIA-mediated pathway in a subject having or susceptible to the disease, disorder or condition.

8. The method of claim 7, wherein the disease, disorder or condition is selected from the group consisting of: cataracts, cancer, Crohn's disease, rheumatoid arthritis, myopathies, inflammatory bowel disease, asthma, coronavirus infection, atherosclerosis, chronic pain and HIV infection.

9. The method of any one of claims 2-8, wherein the inhibitor is any one or more of a small molecule, a peptide, a protein, or an antibody or fragment thereof.

10. The method of claim 9, wherein the inhibitor is a peptide.

11. The method of claim 10, wherein the peptide is a cyclic peptide.

12. The method of claim 11, wherein the cyclic peptide is cyclo-((2-Nal)-Leu-Ser-(2-Nal)-Arg).

13. Use of an inhibitor of the binding of vimentin to hGIIA at coil 2 of vimentin in the manufacture of a medicament for the treatment of a disease, disorder or condition involving a vimentin-mediated pathway and/or a hGIIA-mediated pathway.

14. The use of claim 13, wherein the inhibitor binds to one or more amino acids within amino acid residues 347 to 357 of vimentin according to the amino acid numbering set out in SEQ ID NO: 3.

15. The use of claim 14, wherein the inhibitor binds to one or more of the following amino acids of vimentin according to the amino acid numbering set out in SEQ ID NO: 3: M347, N350, F351, V353, E354 or N357.

16. The use of any one of claims 13-15, wherein the disease, disorder or condition is or comprises inflammation.

17. The use of any one of claims 13-15, wherein the disease, disorder or condition is or comprises epithelial-to-mesenchymal transition.

18. The use of any one of claims 13-15, wherein the disease, disorder or condition is selected from the group consisting of: cataracts, cancer, Crohn's disease, rheumatoid arthritis, myopathies, inflammatory bowel disease, asthma, coronavirus infection, atherosclerosis, chronic pain and HIV infection.

19. The use of any one of claims 13-18, wherein the inhibitor is any one or more of a small molecule, a peptide, a protein, or an antibody or fragment thereof.

20. The use of claim 19, wherein the inhibitor is a peptide.

21. The use of claim 20, wherein the peptide is a cyclic peptide.

22. The use of claim 21, wherein the cyclic peptide is cyclo-((2-Nal)-Leu-Ser-(2-Nal)-Arg).

23. An inhibitor of the binding of vimentin to hGIIA at coil 2 of vimentin, for use in treating a disease, disorder or condition involving a vimentin-mediated pathway and/or a hGIIA-mediated pathway.

24. The inhibitor for the use of claim 23, wherein the inhibitor binds to one or more amino acids within amino acid residues 347 to 357 of vimentin according to the amino acid numbering set out in SEQ ID NO: 3.

25. The inhibitor for the use of claim 24, wherein the inhibitor binds to one or more of the following amino acids of vimentin according to the amino acid numbering set out in SEQ ID NO: 3: M347, N350, F351, V353, E354 or N357.

26. The inhibitor for the use of any one of claims 23-25, wherein the disease, disorder or condition is or comprises inflammation.

27. The inhibitor for the use of any one of claims 23-25, wherein the disease, disorder or condition is or comprises epithelial-to-mesenchymal transition.

28. The inhibitor for the use of any one of claims 23-25, wherein the disease, disorder or condition is selected from the group consisting of: a cataract, a cancer, Crohn's disease, rheumatoid arthritis, a myopathy, an inflammatory bowel disease, asthma, a coronavirus infection, atherosclerosis, chronic pain and a HIV infection.

29. The inhibitor for the use of any one of claims 23-28, wherein the inhibitor is any one or more of a small molecule, a peptide, a protein, or an antibody or fragment thereof.

30. The inhibitor for the use of claim 29, wherein the inhibitor is a peptide.

31. The inhibitor for the use of claim 30, wherein the peptide is a cyclic peptide.

32. The inhibitor for the use of claim 31, wherein the cyclic peptide is cyclo-((2-Nal)-Leu-Ser-(2-Nal)-Arg).

33. A method of screening for an inhibitor of the vimentin-hGIIA interaction, comprising identifying an agent that inhibits the direct physical binding of vimentin to hGIIA.

34. The method of claim 33, comprising identifying an agent that inhibits the binding of vimentin to hGIIA at coil 2 of vimentin.

35. A method of identifying an agent that inhibits the vimentin-hGIIA interaction, comprising contacting coil 2 of vimentin with a test agent, wherein if the test agent binds to coil 2 of vimentin, the test agent is identified as an agent that inhibits the vimentin-hGIIA interaction.

36. A method of screening for a potential inhibitor of inflammation, cellular proliferation and/or epithelial-to-mesenchymal transition, the method comprising determining whether a test agent inhibits the binding of vimentin to hGIIA at coil 2 of vimentin, wherein if the test agent inhibits the binding of vimentin to hGIIA at coil 2 of vimentin, the test agent is identified as a potential inhibitor of inflammation, cellular proliferation and/or epithelial-to-mesenchymal transition.

37. The method of any one of claims 33-36, wherein the agent inhibits a direct physical association between vimentin and hGIIA.

38. The method of any one of claims 33-37, wherein the binding of vimentin to hGIIA is determined at least in part by an enzyme immunoassay.

39. The method of any one of claims 33-38, wherein the method further comprises determining whether the test agent inhibits inflammation, cellular proliferation and/or epithelial-to-mesenchymal transition.

40. The method of claim 39, comprising determining whether the test agent reduces the level of expression or activity of one or more physiological markers of inflammation, cellular proliferation and/or epithelial-to-mesenchymal transition in a test organism.

41. The method of claim 40, wherein the one or more physiological markers of inflammation comprise a prostaglandin.

42. The method of any one of claims 33-41, wherein the agent is any one of:
i) a small molecule compound; or
ii) a peptide; or
iii) a protein; or
iv) an antibody or fragment thereof.

43. The method of any one of claims 33-42, further comprising isolating and/or purifying an agent that inhibits the binding of vimentin to hGIIA.

44. The method of claim 43, further comprising formulating the isolated and/or purified agent into a pharmaceutically acceptable formulation.

45. The method of claim 44, further comprising sterilising the formulation.

46. The method of any one of claims 43-45, further comprising filling the formulation into a container.

47. The method of claim 46, wherein the container is any one or more of a vial, ampoule, bag, blister pack, bottle, cartridge, injection needle, injection syringe, single dose container, strip of multiple single dose containers, or tube.

48. An inhibitor of the vimentin-hGIIA interaction, obtained by the method of any one of claims 33-47.

49. An inhibitor of the vimentin-hGIIA interaction, wherein the inhibitor binds to residues 347 to 357 in coil 2 of vimentin, and wherein the inhibitor is not the cyclic peptide cyclo-((2-Nal)-Leu-Ser-(2-Nal)-Arg).

50. The inhibitor of claim 49, wherein the inhibitor is not a cyclic peptide.

51. The inhibitor of claim 49 or claim 50, wherein the inhibitor is not a peptide.

52. The inhibitor of any one of claims 49-51, obtained by the method of any one of claims 33-47.

53. The inhibitor of any one of claims 49-52, wherein the inhibitor is any one of:
i) a small molecule compound; or
ii) a peptide; or
iii) a protein; or
iv) an antibody or fragment thereof.
